(19) United States
(12) Patent Application Publication
(10) Pub. No.: US 2002/0160527 A1
(43) Pub. Date: Oct. 31, 2002

(54) Combinatorial library comprising pouches as packages for library members and method therefor

(75) Inventors: Jeffrey J. Cernohous, Hudson, WI (US); JingJing Ma, Woodbury, MN (US); Michael P. Daniels, Inver Grove Heights, MN (US); Stephen B. Roscoe, St. Paul, MN (US); Lester H. McIntosh III, Welch, MN (US); Steven D. Koecher, New Brighton, MN (US); Brook F. Duerr, Lake Elmo, MN (US); William H. Sikorski JR., White Bear Lake, MN (US)

Correspondence Address: Attention: Melanie Gover, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/793,666

(22) Filed: Feb. 26, 2001

(51) Int. Cl. G01N 33/545

(52) U.S. Cl. 436/518; 436/85

(57) ABSTRACT

A combinatorial array comprises fluid-impervious, sealed or sealable, flexible, self-supported pouches, each pouch comprising therein one or more members of a library of materials. A method for producing the members of the library is also disclosed. The method can utilize an automated process for introducing components into the pouches. Analysis of products can be by destructive or non-destructive methods.

### FIELD OF THE INVENTION

A combinatorial library comprises sealed flexible self-supported pouches to produce and contain members of the library.

### BACKGROUND OF THE INVENTION

Production of combinatorial arrays involves a research method that focuses on both the creation of massive numbers of samples that make up a library and the rapid screening of these samples in some evaluation designed to determine the efficacy of each sample. The method relies on speed and thoroughness and has already constituted a revolutionary technology in the pharmaceutical industry. Aspects of the method have also been applied to materials and process research.

More particularly, combinatorial synthesis as a separate field of research began with the solid phase synthesis of oligopeptides on an array of solid polymer pins (PCT International Publication No. WO 84/03504). This approach rapidly developed into the synthesis of different compounds onto small polymeric spheres, which could be subjected to a split-and-mix synthesis (see, for example, Acc. Chem. Res. special issue 1996, 29(3), 144-154.). Using this approach, libraries of 10⁶, or more, discrete molecules could be formed and screened for biological activity. There are marked weaknesses to these methods, however, not least of which is the need to chemically bind the molecule of interest to a surface during the synthesis. Although there has been noted success with the synthesis of arrays of biological polymers attached to surfaces (U.S. Pat. No. 5,143,854), the technique lacks generality for materials applications. Furthermore, extended array solids such as ceramics cannot be produced by such a surface-supported route. Other standard combinatorial chemistry techniques, in which a wide variety of different molecules are prepared in a single container and then screened for the desired property (typically biological activity) are inapplicable for the synthesis of materials where the response of individual components would be virtually impossible to identify for a large library. In addition, the properties of materials are typically the properties of assemblages of molecules or atoms or ions and as such the properties of individual components may not be reflected in the properties of a complex mixture.

Research on the parallel synthesis of materials has focussed on thin film syntheses. See e.g., J. J. Hanak, J Mater. Sci. 1970, 5, 964-971; U.S. Pat. No. 5,985,356 and WO 00/04362. Such methods are of limited utility, since many materials cannot be reliably prepared from vapor phase precursors, or cannot be processed to yield the same properties that would be exhibited from a macro scale synthesis. WO98/36826 and WO99/52962 describe macro scale preparations of inorganic and organic materials, respectively. However, these methods involve the synthesis of library members on a small scale, and in a manner that will necessarily differ from the industrial preparation of identified materials in important respects (residence times, heat flow, etc.). The importance of processing parameters to the final properties of commercial materials produced on a large scale is appreciated in industry.

The exploration of synthesis conditions for a library of materials has been addressed by varying the conditions on a single substrate (U.S. Pat. No. 5,345,213, U.S. Pat. No. 5,356,756) or on multiple substrates (U.S. Pat. No. 6,004,617). However, in all these cases the samples are prepared as thin films. Commercial instrumentation is available for evaluating different processing conditions e.g., Argonaut Nautilus organic synthesizer (Argonaut Technologies, San Carlos Calif.). However, samples are once again prepared on a small scale and the problems associated with scale-up remain.

One-dimensional arrays of chemical compounds are known (WO 99/42605) in which the compound is synthesized on an elongated support (string) and the frequency with which each component appears is used for identification. WO 99/32705 describes a string of pouches, each of which is intended to contain a different compound. The pouches are composed of microfilamentous polypropylene to allow the permeation of fluids, and are also radiation treated so that the library elements can be attached to the pouch surface. Various non-pouch designs have also been proposed for supporting molecular libraries on tapes (WO 00/15653, GB 2,295,152).

### SUMMARY OF THE INVENTION

Briefly, the present invention provides a combinatorial array comprising fluid-impervious, flexible, self-supported pouches, each pouch comprising therein one or more members of an organized library of materials.

In another aspect, the present invention provides a method for the synthesis of a combinatorial library of materials comprising the steps of:

a) providing a plurality of fluid-impervious, flexible, self-supported pouches, each pouch comprising therein components for producing one or more members of a combinatorial library of materials,

b) exposing the pouches to a controlled environment to cause the components to interact so as to produce the combinatorial library of materials, and

c) optionally, analyzing the members of the produced library of materials in one or both of non-destructive and destructive processes.

In the method of the invention, components of a reaction mixture for producing members of a combinatorial library can be added simultaneously or sequentially into a flexible pouch. The pouches, which are self-supporting and preferably of unitary construction, can be temporally spaced with respect to each other. A chemical or physical reaction occurs as each individual sealed pouch passes through a specific reaction zone preferably in linear fashion. The sealed pouch provides a barrier to the external environment and provides a package for producing, analyzing, and storing each member of the library of materials. Preferably, in most embodiments, members of the library are not attached to the interior of the pouch surface.

The samples which are separately contained in pouches can be screened in situ by, for example, IR (infrared) spectroscopy, far-IR spectroscopy, UV (ultraviolet) spectroscopy, impedance measurements, ultrasonics, and the like. In addition, such samples can be labeled, (e.g., with a bar code), optionally separated from other pouches, and can be labeled and archived individually or as a plurality of pouches for subsequent further reaction or analysis.

In this application:

“actinic radiation” means electromagnetic radiation, preferably UV, microwave, and IR;

“alloy” means a homogeneous mixture of components;

“blend” means a heterogeneous mixture of components;

“captive” pouch means a pouch smaller than a primary pouch and enclosed therein;

“chemical binding” means a covalent or ionic bond or other chemical linkage;

“combinatorial chemical array” means a matrix or library of pouches, the contents of which are produced by chemical reaction of components to produce, for example, compounds or polymers;

“combinatorial physical array” means a matrix or library of pouches, the contents of which are produced by physical reaction such as by blending, mixing, or alloy formation of components;

“flexible” means can be bent around a rod of diameter 10 cm, preferably 2 cm, more preferably 1 or 2 mm, most preferably 0.25 mm or less;

“film” means a sheet-like material suitable for making into a pouch;

“impervious” means insufficient transport through the pouch to interfere with the reaction during the time of the process;

“macro scale” means reaction mixtures of approximately 0.1 g, preferably 0.5 g, most preferably 1.0 g and up to quantities suitable for commercial production;

“physical binding” means a physical attaching means such as clips, tape, adhesive, etc.;

“pouch” means a flexible, self-supported bag, package, or reaction vessel made of a film that preferably is inert to materials within it and impervious to fluids in the surrounding environment; preferably it is of unitary construction, although a combination of compatible materials can be used;

“primary pouch” means a pouch comprising therein one or more distinct members of a combinatorial library or precursors therefor, and optionally one or more captive pouches;

“radiant energy” means actinic radiation, visible radiation, e-beam, gamma ray, X-ray, and the like”;

“self-supported pouches” means free-standing individually or as a plurality of pouches and not chemically attached to a support, although it can be transported by a conveyance;

“separated temporally” means passing a given point at a different time, i.e., sequentially, as in a linear array; and

“unitary construction” means of one material, except where a septum is present, the septum can be of a different material.

There are significant advantages to using the process of the present invention over traditional high throughput synthetic processes exemplified, for example, in U.S. Pat. No. 5,985,356, U.S. Pat. No. 5,677,195 and WO 84/03564. One such advantage is that the method described herein can be performed as a continuous process by adding pouches to the library as desired. As such, the libraries formed with this method can be large in size because they are not confined to the size of available microtitre plates. Because the invention can be a continuous process, unlike typical combinatorial methods, larger reaction vessel volumes can be used at high process rates. In the present invention, library members can be produced in macro scale amounts with members generally in multi-gram amounts, preferably greater than one gram amounts. Materials in the pouches can be as large as ten grams or even one hundred grams and larger.

Another advantage of the current invention is that the reactions performed in the pouches are easily scalable to commercial production, by increasing the size of the pouches used and/or by increasing the number of pouches in which the desired reaction is being performed. Commercial production sized pouches can be of any size, but typically they can be from 13 cm×5 cm to 100 cm×100 cm. Also, a wide range of reaction chemistry is possible in the same type of pouches, i.e., reactions can be based on chemical or physical reactions to form compounds, polymers, or blends and alloys as well as biological species. These reactions can be controlled by the type of energy supplied in the reaction zone (radiant, thermal, mechanical, ultrasonic,etc.). Unlike typical combinatorial synthetic approaches, the process of the invention provides the capability to change or adjust the reaction conditions as well as the length of time each individual pouch is subjected to the reaction conditions. The process of this invention also makes possible the instantaneous addition, subtraction or alteration of individual samples during the reaction process.

### DETAILED DESCRIPTION OF PREFERRED EMBODIMENTS

The present invention provides a new method of preparing libraries of chemically synthesized or physically mixed materials in a high throughput fashion. The libraries synthesized according to this invention can be separated spatially and temporally. By virtue of the type of reaction vessel utilized, these libraries are amenable to storage without further processing. The invention may employ the steps of simultaneously and/or sequentially combining a plurality of components, for example, as exemplified in U.S. Pat. No. 5,985,356, to achieve a library or array. In accordance with the present invention, the members of the library or array are contained in reaction vessels, which are flexible pouches. The individual pouches pass through a reaction or manipulation zone, which allows chemical reaction or physical mixing within each flexible pouch to occur. The packaging material provides an environment that is preferably inert toward the components and provides an external barrier. In many applications, it is preferred that the components and products do not adhere to the pouch surfaces although adhesion can be useful in some embodiments. The contents of the individual flexible pouches can then be analyzed by various non-destructive or destructive methods to determine the extent of reaction or mixing as well as the properties of the materials produced. In the alternative, the pouches can be stored as a library for later retrieval and analysis. Incorporation of labeling techniques such as, but not limited to, bar coding or radio frequency identification (RFID) tags within the described invention will allow for a quick and efficient means of cataloging, storing, and retrieving the libraries of materials synthesized.

Chemical compounds and biological species can be produced using various techniques, for example, solution reactions in which the product of the reaction remains soluble in the reaction medium; suspension reactions, in which the product of the reaction is insoluble, and is suspended, in the reaction medium; or two phase reactions in which the reactants reside in separate phases. In the latter type of reaction, the reaction takes place at the interface of the separate phases. Compounds can be produced by these techniques as is known in the art, see, for example, WO 95/18972 relating to “Systematic Modular Production of Aminimide- and Oxazolone Based Molecules Having Selected Properties” or WO 91/17271 “Recombinant Library Screening Methods”. The method of the present invention is well-suited for exothermic chemical reactions, because the high surface to volume ratio allows for efficient heat dissipation.

Polymer synthesis methodologies that are accessible using this invention include anionic, cationic, carbo-cationic, free radical, group transfer and coordination catalysis. These methodologies can be accomplished using the polymerization techniques analogous to those used for the synthesis of chemical compounds and biological species, for example, solution polymerization, suspension polymerization, and precipitation polymerization, in which the product of the reaction is insoluble in the reaction medium due to its composition or molecular weight and as such precipitates above a certain threshold concentration. A further method is emulsion polymerization in which the final products are small enough to form a latex or dispersion.

Additionally, adjuvants, which can be used to modify the compounds, biological species, polymers, or blends and alloys of polymers produced, may be included with the initial components or added when desired. These adjuvants can be, but are not limited to, tackifiers, viscosifiers, fillers, chain transfer agents, anti-oxidants, crosslinkers, antimicrobials, compatibilizers or UV stabilizers. Examples of these are the use of glycerol and pentaerythritol esters as tackifiers for the synthesis of adhesives compounds (U.S. Pat. No. 5,257,491), or the use of ascorbic acid as an antioxidant for biological species (Pharm. Res. 2000, 17, 999-1006). In addition, it is well known that sulfur compounds such as butyl mercaptan can be used as chain transfer agents to control the molecular weight of polymers produced using free radical chemistry, see, for example, U.S. Pat. No. 5,932,675. Adjuvants can be added in amounts sufficient to achieve the desired modification of properties. For example, chain transfer agents are typically used in amounts from about 0.001 part to about 10 parts by weight to 100 parts of total monomer when producing copolymerizing acrylic or methacrylic esters; see, for example, U.S. Pat. No. 5,804,610.

Forming a flexible pouch can be accomplished in various ways, for example, heat sealing two lengths of a thermoplastic film together across the bottom and on each lateral edge on a device such as a liquid form-fill-seal machine (for example, using Model 70A2C from General Packaging, Houston Tex.) or manually to form an open ended pouch. Also, a single length of film can be folded and sealed on two edges, charged with components and the remaining edge sealed. Alternatively, a tube of film can be sealed at one end, charged with components and sealed at the opposite end. Pouches can be of any shape that is useful but pouches having rectangular or square surfaces are preferred.

Generally, after the components are introduced into a pouch, it is heat sealed to completely surround the components. The sealing temperature is generally above the softening point and below the melting point of the film used to form the pouch. Removal of most of the air from the pouch prior to sealing is preferred. This may be done by, for example, evacuation or mechanical compression. Seals can be affected in any of a number of different configurations to form multiple pouches across and down the length of the film. For example, in addition to seals on the lateral edges, a seal can also be formed down the center of the film, which, upon sealing of the top and bottom edges, will form two packages. The packages can be left attached to each other by the center seal or cut into individual pouches. In another embodiment, one or a plurality of pouches, herein referred to as captive pouches can be included inside the original pouch in order to add additional components. This can be accomplished either by pre-sealing the additional components into one or more smaller separate captive pouches which can be included during the charging of the initial components or they can be incorporated as smaller internal pouches inside the original pouch. The captive pouches can be free floating or they can be presealed into one or more edges of the primary pouch. The captive pouches containing additional components can be made of material that allows rupture more easily than the primary pouch, effecting contact of the additional components with the primary components. Forming the captive pouches of thinner material than the primary pouch or by utilizing a laminated pouch with a lower melting point facilitates rupturing of the captive pouches. In the former case, the captive pouches can then be ruptured by mechanical agitation such as kneading or compression. In the latter case, an elevated temperature preferably coupled with mechanical agitation can cause rupture of the captive pouches. In an alternative embodiment, captive pouches can be made of a material that decomposes under actinic energy (or other types of energy), which causes the pouch to rupture and release its contents. In another embodiment, the primary pouch can be fitted with a septum inlet to allow resealable entry into the pouch for charging additional components and for removal of samples for analysis of the product, without disturbing the integrity of the pouch for storage.

Pouches preferably comprise a flexible film, which can be UV or IR transparent in certain embodiments. Thermoplastic films are available from many commercial sources, for example, Huntsman Packaging, Rockford Ill. The specific thermoplastic film utilized will depend to a large extent on the composition and melting point of the components and products contained within the pouch, with the softening point of the film generally being less than 125° C. Single layer or multi-layer laminated pouches can be made of flexible thermoplastic polymeric film such as homo- and copolymers of polyolefins, polydienes, polystyrenes, polyesters, polyethers, halogenated polyolefins, polyvinylalcohol, polyamides, polyimines, polycycloolefins, polyphosphazines, polyacetates and polyacrylates. Preferred thermoplastic film materials include low density polyethylene (LDPE), linear low density polyethylene (LLDPE), polypropylene (PP), polyethyleneterephthalate (PET), polytetrafluoroethylene (PTFE), polyvinylidenefluoride (PVF), polyvinylacetate (PVA), copolymers of ethylene and vinyl acetate, vinylidene fluoride, vinyl chloride, teterafluoro ethylene and propylene. Sheets of film are commercially available as noted above, and they can be useful in producing packaged members. Such pouches useful for the combinatorial libraries of the present invention are disclosed for example, in U.S. Pat. No. 5,902,654, incorporated herein by reference for this purpose. Methods for preparing viscoelastic compositions (e.g., adhesives such as hot melt adhesives) in which a pre-viscoelastic composition (e.g., a pre-adhesive composition) is combined with a packaging material and then polymerized by transmissive energy are disclosed in U.S. Pat. Nos. 5,804,610 and 5,932,298, which are incorporated herein by reference for these methods and compositions. A process that involves the packaged polymerization of olefinic monomer(s) and catalyst systems comprising a transition metal species that mediates the polymerization of the monomer(s) is disclosed in U.S. Pat. No. 5,902,654, which is incorporated herein by reference for the process and compositions. This process provides a way to use the resultant polymer without extensive further processing. Other films that can be useful in the present invention include metal films, for example, foils of copper and aluminum and any metal in Groups 2, 3, 4, 5, 6, 7 and 8 on the Periodic Table, as well as composite materials that combine polymer films, metal foils, paper materials, and woven and nonwoven textile materials such as cotton, wool, fiberglass, and polymer fibers.

The thickness of the film utilized for the primary pouch generally varies between about 5 μm-3 mm, preferably 25-250 μm, more preferably 50-150 μm. The thickness of the film also varies depending on the temperature or conditions to which the components of the pouch are to be subjected, with thicker films utilized for high and low temperature applications or applications requiring mechanical manipulation. Captive pouches can be formed of the same or different material and can be the same thickness as the primary pouch or they can be thinner, preferably between about 1 μm -1 mm, more preferably 5-150 μm, most preferably between 15-50 μm. The size of the pouch can be of any desired dimensions. However, persons skilled in the art will recognize that the dimensions of the pouch enables control of the reaction conditions within the pouch to be accomplished. For example, bulk reactions, due to their concentrated mass, require pouches of smaller dimensions than do solution or suspension reactions. This is due to the higher concentration of reacting species and the need for larger surface area to remove thermal energy generated during typical chemical reactions. Solution and suspension reactions on the other hand contain lower concentrations of reacting species and as such require less surface area for thermal energy removal. Primary pouch dimensions for bulk reactions can be of varying sizes, but are generally less than about 100 cm×100 cm, preferably less than about 20 cm×20 cm, more preferably about 13 cm×7 cm or even 2 cm×1 cm or less. The size of the captive pouches adheres to the same constraints and may be of any size provided that it fits within the primary pouch. One skilled in the art will recognize that the type of additional component(s) added from the captive pouches may dictate the size of the primary pouches. For example, if an additional component is a catalyst, the size of the captive pouch required may be quite small in size, e.g., 1 cm×1 cm, whereas if the captive pouch contains a comonomer for a solution copolymerization, the captive pouch may be quite large, e.g., for example, 50 cm×50 cm or less, preferably 10 cm×10 cm or less, most preferably from about 4 cm×5 cm to about 5 mm×5 mm.

Pouches containing components can be linearly and/or horizontally attached to each other or physically separated from each other. After sealing, they can be conveyed through a reaction zone, which can subject each pouch to the same or differing reaction conditions and dwell times. This substantially increases the scope and number of reactions that can be encompassed in an individual library. The reaction zone can be as simple as a constant temperature water bath or as elaborate as a controlled temperature ultrasonic bath. Typically, the duration of reaction time for each pouch can be controlled by the length of the reaction zone utilized. Longer reaction times can require longer reaction zones. Mixing of the components within the pouches can be effected by, but is not limited to, mechanical agitation, e.g., kneading rollers, or controlled pressure gradient changes within a sealed bath, or ultrasonic agitation.

More specifically, the reaction zone can be a liquid, gaseous or solid bath used to initiate and promote chemical or physical reactions and/or control temperature. Formation of the library arrays of the invention, as by chemical or physical reactions, can be facilitated by a variety of energy means, including but not limited to actinic radiation, including thermal, mechanical or ultrasonic energy. Examples of reaction zone baths include but are not limited to water baths, convection ovens, salt baths, and fluidized beds. After passage through the reaction zone, the pouches optionally can be separated and subject to various evaluations or stored for later evaluation and analysis.

In one embodiment of the invention, the separate, self-supported pouches can be placed into and removed manually from one or more reaction zones. In this embodiment, while the process is not mechanically continuous, the products obtained can be subjected to the same constraints as in the following embodiments in that individual pouches can be subject to differing reaction zone conditions and dwell times.

In a preferred alternative embodiment the primary pouches can be separate, free standing, self-supported entities which are temporally spaced with respect to each other. They can be supported by or fastened individually, for example, by means of pins or clamps to a conveyance apparatus such as a moving belt or track for transportation through a reaction zone. This can be a continuous process, wherein, by changing the conditions of the reaction zone (for example temperature, radiant energy, mechanical energy, ultrasonic energy, etc.) and by varying the time spent in the reaction zone, reaction conditions can be varied with each individual pouch, if so desired.

In a most preferred embodiment, the pouches can be joined to each other at one or more edges linearly and/or horizontally. As mentioned above they can be supported by or fastened to a conveyance apparatus. In this embodiment, the pouches are also temporally spaced with respect to each other and can be transported through the reaction zone by various means including rollers, belts, or by rolling onto a spool. Once again, this can also be a continuous process wherein the conditions and duration of time spent within the reaction zone can be varied for each individual pouch if so desired.

Once the pouch has been removed from the reaction zone and the chemical or physical reaction has taken place, the contents of the pouch can be analyzed using techniques that are either destructive or non-destructive to the integrity of the pouch. Non-destructive techniques include analyses that can be performed on the contents of the pouch, through the pouch, without piercing or opening the pouch. Examples of non-destructive techniques include analyses using IR, UV, visible or Raman spectroscopy, refractive index, and acoustical measurements. Physical methods such as compression testing can also be used. Destructive techniques include but are not limited to sampling for nuclear magnetic resonance (NMR), gel permeation chromatography (GPC), differential scanning calorimetry (DSC), thermogravimetric analysis (TGA), dynamic mechanical analysis (DMA), X-ray diffraction (XD), and mass spectral analysis (MS), and the like. For these techniques the pouch must be opened and a sample removed. After a sufficient amount of sample has been taken to complete the desired analyses, the pouch can be resealed. In an alternative embodiment, the sample can be removed, for example, via syringe through a septum incorporated into the pouch or the pouch can be directly pierced with the syringe and the sample withdrawn. In the latter case the pouch can then be resealed using, for example, a pressure sensitive tape or a small amount of adhesive, or heat sealing.

In one embodiment, a library of compounds or materials can be produced, for example, by providing a variety of combinations of components in different ratios in individual pouches for entrance to a reaction chamber and then passing the components through the reaction chamber where reaction occurs.

In a preferred embodiment, the samples may be separately contained within pouches which may, or may not, be attached to one another during their passage through a reactor. If larger samples of some or all elements of a library are required, then the same sample can be reproduced in sequential similarly-sized pouches until the desired quantity is achieved. They may be carried through the reactor supported on or affixed to a mechanical conveyor, or supported on a fluid stream or by some other comparable technique.

The progress of the reaction (or some other property of interest such as polymer molecular weight, etc.) may be evaluated by a technique which can penetrate the containers (optical, spectroscopic, etc.) or the containers may be opened and the contents sampled. Techniques that do not require the containers to be opened are of particular value for in-process screening.

The pouches can be labeled and archived separately, or they can remain attached to each other. Since the containers can be separated, the label is a marker of the identity of the sample contained within. It is not necessarily representative of the position of that sample in any kind of array.

In the method of the present invention sequential reactions can be undertaken by a variety of procedures,

using resealable pouches, such as Ziploc™ bags (SC Johnson, Racine WI) with an appropriate delivery system

using a ruptureable internal captive pouch that under the application of energy (heat, irradiation, mechanical work, chemical energy, etc.) releases a further component for subsequent reaction

using swellable polymers which either with or without the application of energy (heat, irradiation etc.) may release a further component.

This invention discloses creation of libraries useful in organic synthesis, photochemistry, polymer synthesis, and synthesis of biological species. The method is differentiated from other known combinatorial methods in that it provides a linear and or horizontal array of library samples preferably in quantities of 0.5 g up to and including commercially useful quantities, in flexible, impervious, sealable or sealed pouches.

The method is applicable to the large-scale production of commercial materials. The technique will be exemplified by manual creation of one pouch containing a formulation followed by a second pouch containing a different formulation and so on. It preferably can utilize an automated process in which filling of each pouch with reactants, monomers, etc., can be varied using automatic dispensing systems and the pouches can be connected together. Such automatic methods for combining components are disclosed, for example, in U.S. Pat. No. 5,902,654, the methods being incorporated herein by reference.

Objects and advantages of the invention are further illustrated by the following examples, but the particular materials and amounts thereof recited in these examples, as well as other conditions and details, should not be construed to unduly limit this invention.

### EXAMPLES

This invention is further illustrated by the following examples, which are not intended to limit the scope of the invention. In the examples, all parts, ratios, and percentages are by weight unless otherwise indicated. All materials, unless otherwise stated, are available from the Aldrich Chemical Company, Milwaukee Wis. The following test methods were used to characterize the pressure sensitive adhesive compositions in the examples.

### Test Methods

### 180° Peel Adhesion Test

Pressure sensitive adhesive (PSA) samples having a size of 1.25 cm wide and 15 cm long were tested for 180° peel adhesion to a glass substrate. The PSA samples were adhered to the test substrate surface using 6 passes of a 2.1 kg roller. After aging at controlled temperature and humidity conditions (approximately 22° C., 50% relative humidity) for approximately 24 hours, the tapes were tested using a Model 3M90 slip/peel tester (Imass, Inc., Accord, Mass.) in 180° geometry at 30.5 centimeter/minute (cm/min) peel rate, unless otherwise noted.

### Room Temperature Shear Strength Test

Shear strength, as determined by holding time, was measured on PSA samples at controlled temperature and humidity conditions (approximately 22° C., 50% relative humidity). Samples were aged at controlled temperature and humidity conditions (approximately 22° C., 50% relative humidity) for approximately 24 hours. PSA samples having a size of 12.5 mm×12.5 mm were adhered to a stainless steel sheet with 6 passes of a 2.1-kg roller. A 1000-gram weight was then hung from each sample. The amount of time for the weight to drop was recorded. If a sample did not drop, the test was stopped after 10,000 minutes.

### Probe Track Testing

Samples were removed from pouched PSA samples (0.2 g) and were pressed (at 25-50° C. for 10 min) into 0.4 mm thick films on a smooth stainless steel plate surface. Probe tack testing was done using a TA-XT2 Texture Analyzer (Texture Technologies Corp., Scarsdale, N.Y.) with a stainless steel probe (model #57R, 7 mm diameter). In this test, the probe was set to travel downward at a rate of 2.0 mm/sec into the PSA surface until the instrument detected 1.0 g of force. The test was then programmed such that the probe traveled further downward at the test speed of 1.0 mm/s at an applied force of 450 grams for the duration of 0.01 seconds. After this dwell time, the probe was removed from the surface at a rate of 0.5 mm/sec and the peak force and area under the force vs. time curve were calculated and recorded.

### Molecular Weight Characterization

Samples were prepared by the addition of 10 mL of tetrahydrofuran (THF) to approximately 25 mg of sample. The solutions were filtered using a 0.2 μm PTFE syringe filter. 150 μL of solution was then injected into a Polymer Labs PLgel-Mixed B column (Polymer Laboratories, Amherst, Mass.) in a GPC component system consisting of a Waters 717 autosampler (Waters Corp., Milford Mass.) and a Waters 590 pump. The system operated at room temperature, using THF as the eluent, flowing at a rate of 0.95 mL/min. Changes in concentration were detected using an Erma ERC-7515A refractive index detector (Erma CR Inc., Tokyo, Japan). The molecular weight calculations were based upon a calibration made of narrow dispersity polystyrenes ranging in molecular weight from 6.30×10⁶ g/mol to 595 g/mol. The actual calculations were completed with Caliber™ software from Polymer Labs.

### Particle Size Characterization

A Horiba LA-910 dynamic light scattering particle size analyzer with dual helium-neon light sources (Horiba Ltd., Irvine Calif.) was utilized for analyzing emulsion particle sizes. In this technique, approximately 5 mL of a polymerized polymer emulsion was filtered through glass wool into a glass scintillation vial and diluted with deionized water. The sample was placed in a Horiba fraction-cell for analysis and further diluted with deionized water such that 70-95 % transmittance was obtained. The dilution required varied slightly from sample to sample, but was typically on the order of 1000 fold. Once the appropriate transmittance was obtained, the analysis was performed and a mean particle size was determined in microns (μm).

### Gel Testing

2.54 cm×2.54 cm PSA tape samples were punched out and placed into a preweighed wire mesh tray in duplicate. The trays and samples were then weighed and their masses were recorded. The samples were then placed in glass jars and THF (stabilized) was added to a point just below the top of the tray. The jars were capped and the trays containing the PSA tapes were allowed to stand in this solvent for 24 hours. The tray was subsequently placed into a pan and dried in an oven at 70° C. for 10 minutes. The dried pan, tray and sample were then weighed and the percent gel was calculated by determining the residual mass by difference.

### Abbreviations and Tradenames

AA: acrylic acid

AIBN: 2,2′azobisisobutyronitrile, a thermal initiator

ANIS: m-anisaldehyde

BENZ: benzaldehyde

s-BuLi: sec-butyllithium

n-BuOH: 1-butanol

s-BuOH : 2-butanol

CBENZ: 4-chlorobenzaldehyde

CBr₄: carbon tetrabromide, a chain transfer reagent.

DBU: 1,8-diazabicyclo[5.4.0]undec-7-ene, a hindered amine base

DEABENZ: 4-diethylaminobenzaldehyde

DCBENZ: 2,4-dichlorobenzaldehyde

DMA: N,N-dimethylacetoacetamide

EAA: ethylacetoacetate

EBA: ethylbenzylacetoacetate

2-EHA: 2-ethylhexyl acrylate

IOA: isooctyl acrylate

IOTG: isooctylthioglycolate, a chain transfer reagent (Hampshire Chemical Corp., Lexington, Mass.)

Irgacure™ 651: benzyl dimethyl ketal photoinitiator commercially available from Ciba Geigy (Ardsley, N.Y.).

Mazon™ SAM-211: unsaturated poly(alkoxyethyl)sulfate. A polymerizable surfactant available from PPG Industries (Pittsburgh, Pa.).

MeOH: methanol

Mₙ: number average molecular weight

M_{w}: weight average molecular weight

PDI: polydispersity index; M_{w}/Mₙ

PGPE: propylene glycol propyl ether

Piccolastic™ A-75: polystyrene resin available from Hercules (Hercules Inc., Wilmington Del.), with Mₙ=731, PDI=1.77, and softening point=75° C.

PS: polystyrene

SS: stainless steel

### Example 1

### Synthesis and Properties of a Library of Copolymers Produced by Bulk Free Radical Polymerization Using a UV Initiator

Forty-eight 7 cm×11 cm polyethylene pouches were prepared from 0.15 mm thick polyethylene tubing (McMaster Carr, Chicago, Ill.). The pouches were loaded with 18 mL of various mixtures of three different monomers, 0.15 mL of a UV photoinitiator (Darocur™ 1173, Ciba-Geigy), and then heat-sealed. The three monomers chosen were isobornyl acrylate (IBA), 2-EHA, IOTG, and tetrahydrofurfuryl acrylate (THFA). IOTG was used as a chain transfer agent to control molecular weight. The volume ratios for each sample are as shown in Table 1, below:

TABLE 1
actual volumes (mL)

| Sample # | IBA | 2-EHA | THFA | 2-EHA* | Tg (° C.) |
|---|---|---|---|---|---|
| 1 | 6.0 | 3.5 | 6.0 | 2.5 | −18 |
| 2 | 9.0 | 2.0 | 4.5 | 2.5 | −3 |
| 3 | 4.5 | 6.5 | 4.5 | 2.5 | −41 |
| 4 | 4.5 | 2.0 | 9.0 | 2.5 | −20 |
| 5 | 12.0 | 0.5 | 3.0 | 2.5 | 3 |
| 6 | 3.0 | 9.5 | 3.0 | 2.5 | −47 |
| 7 | 3.0 | 0.5 | 12.0 | 2.5 | −27 |
| 8 | 9.0 | 3.5 | 3.0 | 2.5 | −7 |
| 9 | 9.0 | 0.5 | 6.0 | 2.5 | −3 |
| 10 | 3.0 | 6.5 | 6.0 | 2.5 | −38 |
| 11 | 6.0 | 6.5 | 3.0 | 2.5 | −26 |
| 12 | 3.0 | 3.5 | 9.0 | 2.5 | −46 |
| 13 | 6.0 | 0.5 | 9.0 | 2.5 | −10 |
| 14 | 6.8 | 4.3 | 4.5 | 2.5 | −17 |
| 15 | 6.8 | 2.0 | 6.8 | 2.5 | −14 |
| 16 | 4.5 | 4.3 | 6.8 | 2.5 | −33 |
| 17 | 6.0 | 4.0 | 6.0 | 2.0 | −20 |
| 18 | 9.0 | 2.5 | 4.5 | 2.0 | 1 |
| 19 | 4.5 | 7.0 | 4.5 | 2.0 | −33 |
| 20 | 4.5 | 2.5 | 9.0 | 2.0 | −23 |
| 21 | 12.0 | 1.0 | 3.0 | 2.0 | 17 |
| 22 | 3.0 | 10.0 | 3.0 | 2.0 | −46 |
| 23 | 3.0 | 1.0 | 12.0 | 2.0 | −26 |
| 24 | 9.0 | 4.0 | 3.0 | 2.0 | −1 |
| 25 | 9.0 | 1.0 | 6.0 | 2.0 | 5 |
| 26 | 3.0 | 7.0 | 6.0 | 2.0 | −40 |
| 27 | 6.0 | 7.0 | 3.0 | 2.0 | −26 |
| 28 | 3.0 | 4.0 | 9.0 | 2.0 | −34 |
| 29 | 6.0 | 1.0 | 9.0 | 2.0 | −12 |
| 30 | 6.8 | 4.8 | 4.5 | 2.0 | −18 |
| 31 | 6.8 | 2.5 | 6.8 | 2.0 | −13 |
| 32 | 4.5 | 4.8 | 6.8 | 2.0 | −26 |
| 33 | 6.0 | 4.5 | 6.0 | 1.5 | −15 |
| 34 | 9.0 | 3.0 | 4.5 | 1.5 | 1 |
| 35 | 4.5 | 7.5 | 4.5 | 1.5 | −33 |
| 36 | 4.5 | 3.0 | 9.0 | 1.5 | −25 |
| 37 | 12.0 | 1.5 | 3.0 | 1.5 | 15 |
| 38 | 3.0 | 10.5 | 3.0 | 1.5 | −45 |
| 39 | 3.0 | 1.5 | 12.0 | 1.5 | −27 |
| 40 | 9.0 | 4.5 | 3.0 | 1.5 | 0 |
| 41 | 9.0 | 1.5 | 6.0 | 1.5 | 13 |
| 42 | 3.0 | 7.5 | 6.0 | 1.5 | −40 |
| 43 | 6.0 | 7.5 | 3.0 | 1.5 | −24 |
| 44 | 3.0 | 4.5 | 9.0 | 1.5 | −30 |
| 45 | 6.0 | 1.5 | 9.0 | 1.5 | −10 |
| 46 | 6.8 | 5.3 | 4.5 | 1.5 | −19 |
| 47 | 6.8 | 3.0 | 6.8 | 1.5 | −12 |
| 48 | 4.5 | 5.3 | 6.8 | 1.5 | −30 |

*2-EHA is a 0.36 wt % solution of IOTG in 2-EHA

The set of samples was then placed on a carriage and each member was passed sequentially through a temperature-controlled bath (16° C.) under UV-A lamps (intensity 3.5 mW/cm²). The rate of movement of samples below the lamps was adjusted so that each sample spent a total of 9.3 min under illumination. Glass transition temperatures (T_{g}'s) of the resultant polymers were determined by differential scanning calorimetry.

The data of this table show the preparation of ternary or higher copolymer libraries, as well as the use of radical initiators and photo-initiation under UV irradiation. It also demonstrates the use of related sub-libraries containing the same monomer ratios with differing amounts of chain transfer agent.

### Example 2

### Determination of Optimal Conditions for Stability of a Library of Blends of Polymers and Compounds

Blends of polymers and compounds can be created in a two dimensional array based on a polymer and incorporation of various amounts of adjuvants, for example, an antioxidant and a UV stabilizer. The polymer is added to each of a series of pouches, followed by the chosen adjuvants. The level of antioxidant added to each pouch is increased within a specified range for each of the series of pouches, while the level of UV stabilizer is decreased, again, within a specified range. These pouches are then sealed and conveyed through the reaction zone either automatically or manually. The reaction zone for this type of experiment consists of a heated water bath containing offset rollers that physically knead the mixtures of polymer and adjuvants in the pouches to promote homogenization. After removing the pouches from the reaction zone, samples are taken from each pouch and tested for stability when subjected to thermal energy, humidity and UV radiation. This process allows the determination of specific adjuvant levels that provide synergistic effects to the polymer in the form of increased stability. The data will show optimal conditions for stability for each polymer.

### Example 3

### Synthesis of a 90 Member Library of 2-ethylhexyl Acrylate/acrylic Acid Copolymers Useful as PSA Compounds

Ninety pouches were filled with varied amounts of 2-EHA, AA, benzyl dimethyl ketal photoinitiator (Irgacure™ 651, Ciba Geigy), and parts IOTG. The matrix utilized for this example is shown in Table 2, below. The filled pouches (made of 150 μm thick polyethylene film (Huntsman Packaging, Rockford Ill.)) were then manually heat sealed at the top in the cross direction to form pouches measuring 3.25 cm by 12.5 cm. The pouches contained 18.3 g of composition. Each pouch was subsequently placed on a continuous, linear belt for processing. In these examples, the pouches were placed on a belt that traversed through a water bath that was maintained between about 21 ° C. and 32° C. and exposed to ultraviolet radiation at an intensity of about 2.0 mW/cm² for 8.33 minutes. The radiation was supplied from lamps having about 90% of its emissions between 300 and 400 nanometers (nm), and a peak emission at 351 nm. The resulting pouched samples were hot-melt compounded and coated using the following method:

The pouched PSAs were placed into a heated (240-370 ° C.) section of static mixing elements. A reciprocating piston was used to repeatedly push the samples through the mixing elements and integrally mix the pouched PSAs. Once sufficient mixing had been achieved, the material was coated through a hot (240-370 ° C.) die onto a moving substrate. The substrate speed typically ranged from 2.3-10.7 m/min. Typical coating thickness were in the range of 37.5-50.0 μm, and were directly determined by the melt viscosity of the material and the speed of the moving substrate.

The molecular weight (M_{w}), polydispersity (PDI), and gel % were determined for the pouched PSAs while tack, peel, and shear properties were determined on the resulting coated PSA tapes. The data obtained for each of the samples in this library are given in Tables 3 and 4, below.

TABLE 2

| Sample Matrix | | 2-EHA/AA Weight Ratio | | | | | | | | | |
|---|---|---|---|---|---|---|---|---|---|---|---|
| Wt % | Wt % | 98/ | 96/ | 94/ | 92/ | 90/ | 88/ | 86/ | 84/ | 82/ | 80/ |
| IOTG | Initiator | 2 | 4 | 6 | 8 | 10 | 12 | 14 | 16 | 18 | 20 |
| 0.02 | 0.05 | 1 | 10 | 19 | 28 | 37 | 46 | 55 | 64 | 63 | 82 |
| 0.04 | 0.05 | 2 | 11 | 20 | 29 | 38 | 47 | 56 | 65 | 74 | 83 |
| 0.06 | 0.05 | 3 | 12 | 21 | 30 | 39 | 48 | 57 | 66 | 75 | 84 |
| 0.02 | 1.00 | 4 | 13 | 22 | 31 | 40 | 49 | 58 | 67 | 76 | 85 |
| 0.04 | 1.00 | 5 | 14 | 23 | 32 | 41 | 50 | 59 | 68 | 77 | 86 |
| 0.06 | 1.00 | 6 | 15 | 24 | 33 | 42 | 51 | 60 | 69 | 78 | 87 |
| 0.02 | 1.05 | 7 | 16 | 25 | 34 | 43 | 52 | 61 | 70 | 79 | 88 |
| 0.04 | 1.05 | 8 | 17 | 26 | 35 | 44 | 53 | 62 | 71 | 80 | 89 |
| 0.06 | 1.05 | 9 | 18 | 27 | 36 | 45 | 54 | 63 | 72 | 81 | 90 |

TABLE 3

| Sample | EHA/AA | % | Tack | Tack - total | Peel test | Shear test |
|---|---|---|---|---|---|---|
| # | wt ratio | gel | (g) | energy (g · s) | (N/m) | (min) |
| 1 | 98/2 | 0.2 | 284 | 536 | 27.4 | 0.1 |
| 2 | 98/2 | 1.5 | 236 | 321 | 16.7 | 0.1 |
| 3 | 98/2 | 1.9 | 235 | 222 | 10.6 | 0.0 |
| 4 | 98/2 | 3.0 | 287 | 602 | 34.2 | 0.1 |
| 5 | 98/2 | 2.5 | 264 | 452 | 21.5 | 0.1 |
| 6 | 98/2 | 2.3 | 231 | 275 | 16.6 | 0.1 |
| 7 | 98/2 | 2.4 | 279 | 571 | 43.0 | 0.1 |
| 8 | 98/2 | 3.1 | 240 | 439 | 23.5 | 0.1 |
| 9 | 98/2 | 2.4 | 251 | 244 | 12.7 | 0.1 |
| 10 | 96/4 | 1.8 | 308 | 873 | 11.9 | 0.3 |
| 11 | 96/4 | 2.1 | 319 | 581 | 71.0 | 0.1 |
| 12 | 96/4 | 2.0 | 292 | 563 | 71.5 | 0.1 |
| 13 | 96/4 | 2.4 | 316 | 992 | 12.2 | 0.4 |
| 14 | 96/4 | 1.5 | 349 | 844 | 14.4 | 0.3 |
| 15 | 96/4 | 1.1 | 333 | 387 | 45.6 | 0.1 |
| 16 | 96/4 | 1.7 | 334 | 752 | 23.1 | 0.4 |
| 17 | 96/4 | 0.6 | 315 | 471 | 35.8 | 0.3 |
| 18 | 96/4 | 2.0 | 338 | 369 | 57.0 | 0.1 |
| 19 | 94/6 | 1.3 | 413 | 1008 | 20.0 | 1.7 |
| 20 | 94/6 | 2.3 | 386 | 791 | 16.5 | 0.3 |
| 21 | 94/6 | 0.9 | 378 | 1131 | 23.8 | 0.4 |
| 22 | 94/6 | 1.6 | 440 | 1726 | 18.4 | 2.0 |
| 23 | 94/6 | 1.7 | 442 | 1489 | 23.9 | 0.8 |
| 24 | 94/6 | 2.1 | 403 | 1570 | 24.5 | 0.7 |
| 25 | 94/6 | 1.3 | 413 | 1581 | 23.9 | 1.8 |
| 26 | 94/6 | 1.7 | 386 | 1583 | 26.4 | 0.7 |
| 27 | 94/6 | 2.2 | 405 | 959 | 34.8 | 0.3 |
| 28 | 92/8 | 0.5 | 493 | 918 | 18.4 | 2.0 |
| 29 | 92/8 | 1.1 | 439 | 1044 | 17.3 | 1.0 |
| 30 | 92/8 | 0.5 | 485 | 1219 | 17.7 | 0.6 |
| 31 | 92/8 | 0.0 | 528 | 966 | 22.3 | 4.2 |
| 32 | 92/8 | 1.1 | 492 | 912 | 24.3 | 2.9 |
| 33 | 92/8 | 0.3 | 483 | 1825 | 29.2 | 1.7 |
| 34 | 92/8 | 1.5 | 482 | 1109 | 25.9 | 5.0 |
| 35 | 92/8 | 0.0 | 490 | 991 | 25.7 | 3.0 |
| 36 | 92/8 | 0.0 | 489 | 2513 | 25.7 | 1.4 |
| 37 | 90/10 | 0.1 | 602 | 1045 | 19.5 | 11.6 |
| 38 | 90/10 | 0.0 | 564 | 1193 | 18.5 | 5.9 |
| 39 | 90/10 | 0.0 | 615 | 1295 | 21.3 | 3.2 |
| 40 | 90/10 | 0.0 | 604 | 1100 | 25.5 | 14.7 |
| 41 | 90/10 | 0.2 | 594 | 1043 | 24.2 | 9.1 |
| 42 | 90/10 | 0.0 | 574 | 816 | 20.5 | 3.0 |
| 43 | 90/10 | 1.1 | 593 | 905 | 26.9 | 15.2 |
| 44 | 90/10 | 1.3 | 574 | 1284 | 29.9 | 7.5 |
| 45 | 90/10 | 0.6 | 615 | 1322 | 34.3 | 8.4 |

TABLE 4

| Sample | EHA/AA | % | Tack | Tack - total | Peel test | Shear test |
|---|---|---|---|---|---|---|
| # | ratio | gel | (g) | energy (g · s) | (N/m) | (min) |
| 46 | 88/12 | 0.8 | 640 | 533 | 24.9 | 28.3 |
| 47 | 88/12 | 0.9 | 707 | 1258 | 16.3 | 10.4 |
| 48 | 88/12 | 1.2 | 631 | 2240 | 21.0 | 4.6 |
| 49 | 88/12 | 0.7 | 744 | 883 | 27.2 | 55.3 |
| 50 | 88/12 | 0.0 | 604 | 740 | 28.0 | 40.5 |
| 51 | 88/12 | 0.2 | 703 | 1082 | 35.4 | 26.9 |
| 52 | 88/12 | 0.0 | 711 | 667 | 31.4 | 59.6 |
| 53 | 88/12 | 0.3 | 726 | 729 | 31.7 | 24.7 |
| 54 | 88/12 | 0.9 | 695 | 1208 | 27.4 | 9.3 |
| 55 | 86/14 | 0.0 | 698 | 868 | 18.5 | 46.9 |
| 56 | 86/14 | 0.7 | 803 | 327 | 20.0 | 39.4 |
| 57 | 86/14 | 1.2 | 751 | 806 | 25.4 | 28.9 |
| 58 | 86/14 | 0.6 | 791 | 312 | 22.9 | 102.4 |
| 59 | 86/14 | 1.9 | 773 | 641 | 31.0 | 79.9 |
| 60 | 86/14 | 2.1 | 713 | 224 | 24.7 | 38.7 |
| 61 | 86/14 | 0.5 | 820 | 608 | 29.4 | 117.0 |
| 62 | 86/14 | 0.5 | 752 | 241 | 31.4 | 77.4 |
| 63 | 86/14 | 1.1 | 836 | 544 | 35.4 | 45.7 |
| 64 | 84/16 | 1.3 | 707 | 126 | 30.5 | 212.2 |
| 65 | 84/16 | 2.0 | 727 | 349 | 27.4 | 152.7 |
| 66 | 84/16 | 0.9 | 652 | 111 | 29.6 | 121.0 |
| 67 | 84/16 | 0.0 | 769 | 185 | 15.5 | 309.2 |
| 68 | 84/16 | 2.2 | 709 | 241 | 24.0 | 177.9 |
| 69 | 84/16 | 1.4 | 754 | 340 | 30.7 | 118.3 |
| 70 | 84/16 | 1.1 | 688 | 179 | 26.1 | 242.1 |
| 71 | 84/16 | 0.0 | 710 | 758 | 29.9 | 115.5 |
| 72 | 84/16 | 0.5 | 842 | 382 | 26.5 | 103.1 |
| 73 | 82/18 | 1.3 | 714 | 160 | 27.0 | 243.6 |
| 74 | 82/18 | 4.3 | 679 | 137 | 28.6 | 212.8 |
| 75 | 82/18 | 0.4 | 586 | 130 | 22.4 | 233.4 |
| 76 | 82/18 | 2.0 | 658 | 115 | 9.3 | 689.3 |
| 77 | 82/18 | 0.9 | 516 | 87 | 11.6 | 527.7 |
| 78 | 82/18 | 1.2 | 542 | 81 | 6.0 | 352.0 |
| 79 | 82/18 | 0.0 | 306 | 34 | 2.0 | 1595.7 |
| 80 | 82/18 | 0.0 | 435 | 53 | 3.2 | 291.8 |
| 81 | 82/18 | 0.0 | 364 | 37 | 3.7 | 975.8 |
| 82 | 80/20 | 0.2 | 356 | 56 | 20.7 | 545.9 |
| 83 | 80/20 | 1.2 | 370 | 35 | 23.3 | 531.5 |
| 84 | 80/20 | 0.5 | 422 | 46 | 16.4 | 332.0 |
| 85 | 80/20 | 0.8 | 320 | 29 | 3.0 | 1304.5 |
| 86 | 80/20 | 0.0 | 423 | 49 | 2.4 | 475.1 |
| 87 | 80/20 | 1.7 | 290 | 23 | 2.7 | 1141.4 |
| 88 | 80/20 | 1.5 | 182 | 15 | 0.6 | 4766.2 |
| 89 | 80/20 | 2.0 | 47 | 2 | 1.4 | 2509.7 |
| 90 | 80/20 | 1.1 | 90 | 4 | 1.1 | 12764.3 |

### Example 4

### Anionic Polymerization in Resealable Pouches to Produce a 20 Member Library of Homopolymers Useful as Thermoplastic Materials

A modified version of the pouch described in example 3 was utilized to anionically polymerize styrene monomer. In this 20 member library, each 100 μm thick polyethylene pouch with dimensions of 6.5 cm×10.0 cm, equipped with a zipper lock seal (about 20 mL total volume) was filled with varied amounts of styrene and cyclohexane. The pouched solution was then purged with argon for 5 minutes, and cooled to 0° C. sec-Butyllithium was subsequently injected into the pouch to initiate the polymerization and the pouch was immediately hand sealed and submerged in an ice-water bath. After 30 minutes, the zipper lock seal was opened and 1-2 mL isopropanol was added into the pouch to quench the polymerization. The sample was then dissolved in THF and the resulting solution was poured into isopropanol with stirring to precipitate the polymer. The polymer slurry was then collected via filtration and dried under vacuum (10 mm Hg) at 60° C. for 2 hours. The sample matrix utilized for this example is given in Table 5, below. The molecular weight (M_{w}) and PDI data that were obtained for each of the samples in this library are also given in Table 5, below.

TABLE 5

| Sam- | | | Volume | Predicted | Actual Mₙ | |
|---|---|---|---|---|---|---|
| ple | Moles | Moles | cyclohex- | Mₙ | (GPC) | PDI |
| # | styrene | s-BuLi | ane (mL) | (kg/mol) | (kg/mol) | (M_{w}/Mₙ) |
| 1 | 0.044 | 0.00013 | 0 | 35 | No Poly- | * |
| | | | | | merization | |
| 2 | 0.044 | 0.00026 | 0 | 17.5 | No Poly- | * |
| | | | | | merization | |
| 3 | 0.044 | 0.00039 | 0 | 11.7 | 15.5 | 2.29 |
| 4 | 0.044 | 0.00052 | 0 | 8.8 | 12.7 | 2.62 |
| 5 | 0.044 | 0.00065 | 0 | 7.1 | 14.2 | 2.71 |
| 6 | 0.044 | 0.00078 | 0 | 5.9 | 7.8 | 2.50 |
| 7 | 0.044 | 0.00091 | 0 | 5.1 | 10.3 | 2.50 |
| 8 | 0.044 | 0.00104 | 0 | 4.4 | 9.3 | 3.39 |
| 9 | 0.044 | 0.00117 | 0 | 3.9 | 8.8 | 2.18 |
| 10 | 0.044 | 0.00130 | 5 | 3.6 | 8.2 | 1.98 |
| 11 | 0.044 | 0.00143 | 5 | 3.2 | pouch | * |
| | | | | | melted | |
| 12 | 0.044 | 0.00156 | 5 | 3.0 | 7.1 | 1.82 |
| 13 | 0.044 | 0.00169 | 5 | 2.8 | 5.6 | 1.57 |
| 14 | 0.044 | 0.00182 | 5 | 2.6 | 5.6 | 1.61 |
| 15 | 0.044 | 0.00195 | 5 | 2.4 | 6.2 | 1.66 |
| 16 | 0.044 | 0.00208 | 5 | 2.2 | 5.2 | 1.57 |
| 17 | 0.044 | 0.00221 | 5 | 2.1 | 4.3 | 1.47 |
| 18 | 0.044 | 0.00234 | 5 | 2.0 | 4.0 | 1.38 |
| 19 | 0.044 | 0.00247 | 5 | 1.9 | 3.7 | 1.36 |
| 20 | 0.044 | 0.00260 | 5 | 1.8 | pouch | * |
| | | | | | melted | |

*Not available

### Example 5

### Anionic Polymerization in Resealable Pouches to Produce a 10 Member of Copolymers Useful as Synthetic Rubbers

A modified version of a pouch described in example 3 was utilized to anionically polymerize styrene monomer. In this 10 member library, each polyethylene pouch with a wall thickness of 100 μm, measuring 6.5 cm×10 cm (about 20 mL pouch volume) and equipped with a zipper lock seal was filled with 10 mL of a 50 wt % solution of styrene in cyclohexane. The pouched solution was then purged with argon for 5 minutes, and cooled to 0° C. sec-Butyllithium was subsequently injected into the pouch to initiate the polymerization and the pouch was immediately hand sealed and submerged in an ice-water bath. After 30 minutes, the zipper lock seal on each pouch was reopened and diphenylethylene was added. The pouch was reclosed for 15 minutes, and the pouch was subsequently reopened and 2-ethylhexyl acrylate was added. The viscous solution was allowed to react for 15 minutes and then the zipper lock seal was opened and 1-2 mL isopropanol was added into the pouch to quench the polymerization. The sample was dissolved in THF and the resulting solution was poured into methanol with stirring to precipitate the polymer. The polymer slurry was then collected and dried under vacuum (10 mm Hg) at 60° C. for 2 hours. The molecular weight (M_{w}), PDI, and compositional data as determined by NMR that were obtained for each of the samples in this library are given in Table 6, below.

TABLE 6

| | | | Theoret- | | Pre- | | |
|---|---|---|---|---|---|---|---|
| | Moles | | ical molar | | dicted | Actual | |
| Sam- | di- | Moles | ratio | Actual | Mₙ | Mₙ | PDI |
| ple | phenyl- | 2- | styrene/ | Molar | (kg/ | (kg/ | (M_{w}/ |
| # | ethylene | EHMA | 2-EHA | Ratio | mol) | mol) | Mₙ) |
| 1 | 0.0013 | 0.0045 | 9.8 | 20.0 | 4.4 | 8.8 | 2.99 |
| 2 | 0.0013 | 0.0089 | 4.9 | 7.1 | 5.1 | 12.3 | 4.80 |
| 3 | 0.0013 | 0.013 | 3.3 | 4.9 | 5.8 | 12.5 | 5.33 |
| 4 | 0.0013 | 0.018 | 2.5 | 3.1 | 6.5 | 16.4 | 5.66 |
| 5 | 0.0013 | 0.022 | 2.0 | 2.4 | 7.1 | 14.0 | 5.22 |
| 6 | 0.0039 | 0.0015 | 9.8 | 8.5 | 4.4 | 8.8 | 3.18 |
| 7 | 0.0039 | 0.0089 | 4.9 | 4.6 | 5.1 | 9.9 | 3.85 |
| 8 | 0.0039 | 0.013 | 3.3 | 2.5 | 5.8 | 12.0 | 5.37 |
| 9 | 0.0039 | 0.018 | 2.5 | 1.5 | 6.5 | 13.4 | 5.36 |
| 10 | 0.0039 | 0.022 | 2.0 | 1.2 | 7.1 | 13.6 | 5.14 |

### Example 6

### Anionic Polymerization to Produce a 20 Member Library of Homopolymers Where one Reagent/monomer Was Sealed in a Captive Pouch Within a Resealing Primary Pouch Containing the Initial Polymerization Components to Produce Polymers Useful as Thermosetting Materials

The 20 members of the library of this example were synthesized in pouches as described in example 3. Additionally, a captive pouch containing the initiator for the polymerization was added into each pouch prior to heat sealing.

In this library, 6.5 cm×10 cm (about 20 ml pouch volume) polyethylene pouches with a wall thickness of 150 μm were filled with varied amounts of styrene and cyclohexane. The pouched solution was then purged with argon for 5 min. Sec-butyllithium was added into a separate polyethylene pouch with a wall thickness of 37.5 μm and dimensions of 4 cm×5 cm ( about 2.5 ml pouch volume) which was heat sealed and placed within the primary pouch. The primary pouch was then heat-sealed and the captive pouch containing the initiator was ruptured with hand pressure to initiate the polymerization, then the pouch was immediately submerged in an ice-water bath. The solution was allowed to react for 30 minutes and then 1-2 mL isopropanol was injected into the pouch to quench the polymerization. The sample was dissolved in THF and the resulting solution was poured into isopropanol with stirring to precipitate the polymer. The polymer slurry was then collected by filtration and dried under vacuum (10 mm Hg) at 60° C. for 2 hours. The sample matrix utilized for this example is given in Table 7. The molecular weight (M_{w}) and PDI data that were obtained for each of the samples in this library are also given in Table 7, below.

TABLE 7

| Sam- | | | Volume | Predicted | Actual | |
|---|---|---|---|---|---|---|
| ple | moles | s-BuLi | cyclohexane | Mₙ | Mₙ | PDI |
| # | styrene | mmoles | mL | kg/mol | kg/mol | (M_{w}/Mₙ) |
| 1 | 0.044 | 0.650 | 0 | 7.1 | 9.2 | 1.98 |
| 2 | 0.044 | 0.975 | 0 | 4.8 | 5.5 | 1.78 |
| 3 | 0.044 | 1.300 | 0 | 3.6 | 6.8 | 1.94 |
| 4 | 0.044 | 1.625 | 0 | 2.9 | 2.7 | 1.46 |
| 5 | 0.044 | 1.950 | 0 | 2.4 | 2.2 | 1.36 |
| 6 | 0.044 | 0.650 | 1.67 | 7.1 | 9.0 | 1.53 |
| 7 | 0.044 | 0.975 | 1.67 | 4.8 | 6.8 | 3.66 |
| 8 | 0.044 | 1.300 | 1.67 | 3.6 | 4.2 | 1.48 |
| 9 | 0.044 | 1.625 | 1.67 | 2.9 | 4.3 | 1.40 |
| 10 | 0.044 | 1.950 | 1.67 | 2.4 | 2.4 | 1.30 |
| 11 | 0.044 | 0.650 | 5.0 | 7.1 | 11.6 | 2.53 |
| 12 | 0.044 | 0.975 | 5.0 | 4.8 | 4.5 | 3.34 |
| 13 | 0.044 | 1.300 | 5.0 | 3.6 | 3.4 | 1.39 |
| 14 | 0.044 | 1.625 | 5.0 | 2.9 | 2.5 | 1.43 |
| 15 | 0.044 | 1.950 | 5.0 | 2.4 | 2.1 | 1.35 |
| 16 | 0.044 | 0.650 | 15.0 | 7.1 | 6.2 | 4.44 |
| 17 | 0.044 | 0.975 | 15.0 | 4.8 | 3.8 | 1.27 |
| 18 | 0.044 | 1.300 | 15.0 | 3.6 | 3.5 | 1.82 |
| 19 | 0.044 | 1.625 | 15.0 | 2.9 | 2.1 | 1.20 |
| 20 | 0.044 | 1.950 | 15.0 | 2.4 | 2.4 | 1.45 |

### Example 7

### Emulsion Copolymerization to Produce a 24 Member Library of Polymers Useful as PSAs

The 24 members of the library of this example were produced in pouches as described in example 3. The conditions utilized for these polymerizations were as described in U.S. Pat. No. 6,048,611, which is incorporated herein by reference. Specifically, in this example, specified amounts of deionized water (147 g), a copolymerizable surfactant (Mazon SAM 211) (5.25 to 15.95 g), isooctyl acrylate (245 g), acrylic acid (8.5 g), vinyl acetate (16.5 g), polystyrene (5.5 g), and carbon tetrabromide (0.55 g) were placed in a 1 L stainless steel Waring blender and emulsified at high speed for 1 minute. In all cases, one drop of a 1% solution of FeSO₄.7H₂O was added and gently mixed to each 100 g of emulsion. A 25 mL volume of each solution was then charged into a 6.5 cm×10 cm (about 25 mL pouch volume) polyethylene pouch with a wall thickness of 150 μm thick. A specified amount of either a redox initiator pair (potassium persulfate/sodium metabisulfite) or a free radical initiator (potassium persulfate) was then charged into the pouch. The pouch was subsequently hand-sealed and placed in a 60° C. water bath for 30 minutes. The temperature of the bath was then increased to 80° C. for an additional 3.5 hours. The resulting solutions were then analyzed for percent solids and particle size. The detailed library and the characterization results are given in Table 8, below. Theoretical % solids were calculated to be 32.6% for all the samples in Table 8, below.

TABLE 8

| Sam- | | | | | Mean |
|---|---|---|---|---|---|
| ple | | Wt % | Wt % | Wt % | particle size |
| # | Initiator system | surfactant | initiator | solids | (μm) |
| 1 | 4/1 K₂S₂O₈/Na₂S₂O₈ | 1.25 | 0.05 | 24.9 | 0.93 |
| 2 | 4/1 K₂S₂O₈/Na₂S₂O₈ | 1.25 | 0.1 | 28.7 | 0.82 |
| 3 | 4/1 K₂S₂O₈/Na₂S₂O₈ | 1.25 | 0.15 | 28.8 | 0.89 |
| 4 | 4/1 K₂S₂O₈/Na₂S₂O₈ | 1.25 | 0.2 | 29.2 | 0.84 |
| 5 | 4/1 K₂S₂O₈/Na₂S₂O₈ | 2.5 | 0.05 | 29.7 | 0.67 |
| 6 | 4/1 K₂S₂O₈/Na₂S₂O₈ | 2.5 | 0.1 | 27.2 | 0.73 |
| 7 | 4/1 K₂S₂O₈/Na₂S₂O₈ | 2.5 | 0.15 | 28.7 | 0.77 |
| 8 | 4/1 K₂S₂O₈/Na₂S₂O₈ | 2.5 | 0.2 | 31.1 | 0.73 |
| 9 | 4/1 K₂S₂O₈/Na₂S₂O₈ | 3.75 | 0.05 | 28.2 | 0.52 |
| 10 | 4/1 K₂S₂O₈/Na₂S₂O₈ | 3.75 | 0.1 | 29.9 | 0.59 |
| 11 | 4/1 K₂S₂O₈/Na₂S₂O₈ | 3.75 | 0.15 | 31.0 | 0.55 |
| 12 | 4/1 K₂S₂O₈/Na₂S₂O₈ | 3.75 | 0.2 | 31.1 | 0.61 |
| 3 | K₂S₂O₈ | 1.25 | 0.05 | 21.1 | 0.75 |
| 14 | K₂S₂O₈ | 1.25 | 0.1 | 24.2 | 0.52 |
| 15 | K₂S₂O₈ | 1.25 | 0.15 | 25.9 | 0.54 |
| 16 | K₂S₂O₈ | 1.25 | 0.2 | 24.4 | 0.54 |
| 17 | K₂S₂O₈ | 2.5 | 0.05 | 28.6 | 0.90 |
| 18 | K₂S₂O₈ | 2.5 | 0.1 | 29.1 | 0.50 |
| 19 | K₂S₂O₈ | 2.5 | 0.15 | 29.1 | 0.54 |
| 20 | K₂S₂O₈ | 2.5 | 0.2 | 28.8 | 0.51 |
| 21 | K₂S₂O₈ | 3.75 | 0.05 | 29.1 | 0.47 |
| 22 | K₂S₂O₈ | 3.75 | 0.1 | 29.8 | 0.47 |
| 23 | K₂S₂O₈ | 3.75 | 0.15 | 29.1 | 0.50 |
| 24 | K₂S₂O₈ | 3.75 | 0.2 | 28.3 | 0.46 |

### Example 8

### Synthesis of an 18 Member Library of Dihydropyrimidines

In this example, eighteen 13 cm×7 cm polyethylene pouches with a wall thickness of 150 μm thick were charged individually with the components shown in Table 9, below, followed by the addition of 0.01 g p-toluenesulfonic acid sodium salt and 12.0 ml methanol.

TABLE 9

| | Compo- | Compo- | Compo- | | ProbeMS Analysis |
|---|---|---|---|---|---|
| | nent 1 | nent 2 | nent 3 | Solids | (Dihydro- |
| Sample | (22.5 | (22.5 | (33.6 | Yield | pyrimidines |
| # | mmol) | mmol) | mmol) | (g) | Product Observed) |
| 1 | urea | BENZ | EAA | 0.01 | Yes |
| 2 | thiourea | BENZ | EAA | 0 | No |
| 3 | urea | BENZ | EBA | 2.13 | No |
| 4 | urea | CBENZ | EAA | 0.91 | Yes |
| 5 | urea | CBENZ | EBA | 3.37 | No |
| 6 | urea | DCBENZ | EAA | 4.43 | Yes |
| 7 | urea | DCBENZ | EBA | 7.16 | No |
| 8 | thiourea | CBENZ | EBA | 0.06 | No |
| 9 | thiourea | DCBENZ | EBA | 0 | No |
| 10 | urea | DEABENZ | EAA | 0.39 | Yes |
| 11 | urea | DEABENZ | EBA | 0 | No |
| 12 | urea | DEABENZ | DMA | 0 | No |
| 13 | urea | BENZ | DMA | 0 | No |
| 14 | urea | ANIS | DMA | 0 | No |
| 15 | urea | CBENZ | DMA | 0 | No |
| 16 | urea | DCBENZ | DMA | 5.15 | Yes |
| 17 | urea | ANIS | EAA | 0.81 | Yes |
| 18 | urea | ANIS | EBA | 0.17 | No |

The pouches were then sealed and the contents were briefly mixed. To simulate a continuous process, the pouches were heat sealed together and placed on a motorized conveyor through a water bath maintained at 60° C. The speed of the conveyor was adjusted such that each individual pouch would be in the constant temperature water bath for 2.5 hours. After all the samples had passed through the water bath, they were allowed to dry and were placed in a freezer at −17° C. for 7 days. The pouches were then opened and the contents washed with excess methanol and filtered. Drying for 18 hours under vacuum (10 mm Hg) at 60° C. yielded the products as solids of various colors. The compounds were analyzed by direct thermal desorption ionization mass spectrometry (Probe MS) using a Zabspec magnetic sector mass spectrometer (Micromass Inc., Beverly, Mass.). Yields and mass spectral analysis data are also shown in Table 9, above.

### Example 9

### Solution Formulation of a 21 Member Library of Polymer Blends and Alloys

In this example, twenty-one 6 cm×7 cm resealable polyvinylacetate pouches with a wall thickness of 150 μm (Anchor Paper, Brooklyn Center, Minn.) were charged individually with the amounts of polystyrene (M_{w}800-5,000, Polysciences Inc. Warrington, Pa.) and polybutadiene (Mₙ5,000, 20% 1-2 addition units, Aldrich Milwaukee, Wis.) shown in Table 10, below, followed by the addition of 10.0 mL THF. The pouches were then heat sealed and mechanically agitated for 4.0 hours. Each pouch was cut open with a pair of scissors and an aliquot (about 0.10 mL) of each mixture was taken and placed into a tared DSC pan and the THF was allowed to evaporate, followed by drying for 18 hours under vacuum (10 mm Hg) at 40° C. The pans were then weighed and sealed, followed by DSC analysis from −100° C. to 150° C. at a heating rate of 10° C./min. DSC analysis was performed on a Dupont 912 differential scanning calorimeter (Dupont, Research Station, DE). The apparent T_{g}s are listed in Table 10, below.

TABLE 10

| | Polybutadiene | Polystyrene | T_{g}s (° C.) | |
|---|---|---|---|---|
| Sample # | wt % | wt % | Polybutadiene | Polystyrene |
| 1 | 0.0 | 100 | * | 58.3 |
| 2 | 5 | 95 | * | 55.0 |
| 3 | 10 | 90 | * | 49.1 |
| 4 | 15 | 85 | −71.6 | 48.9 |
| 5 | 20 | 80 | −75.5 | 50.2 |
| 6 | 25 | 75 | −78.8 | 50.0 |
| 7 | 30 | 70 | −80.2 | 46.7 |
| 8 | 35 | 65 | −80.2 | 49.5 |
| 9 | 40 | 60 | −80.4 | 54.5 |
| 10 | 45 | 55 | −80.2 | 55.7 |
| 11 | 50 | 50 | −79.9 | 54.6 |
| 12 | 55 | 45 | −79.7 | 54.2 |
| 13 | 60 | 40 | −82.0 | 66.5 |
| 14 | 65 | 35 | −83.3 | 66.8 |
| 15 | 70 | 30 | −82.0 | 65.4 |
| 16 | 75 | 25 | −81.9 | 66.03 |
| 17 | 80 | 20 | −81.5 | * |
| 18 | 85 | 15 | −83.3 | * |
| 19 | 90 | 10 | −84.7 | * |
| 20 | 95 | 5 | −86.7 | * |
| 21 | 100 | 0 | −88.3 | * |

*no T_{g} detected

The data in Table 10 show that the samples having between 15 and 80 wt % polybutadiene exhibited 2 T_{g}s (one for both the polybutadiene and polystyrene fractions in each sample), corresponding to an immiscible polymer/polymer blend. The remaining samples (5 to 10 wt % and 80 to 95 wt % polybutadiene), exhibiting only one T_{g}, were indicative of the formation of a miscible polymer/polymer alloy.

### Example 10

### The Use of a Captive Pouch in Orthogonal Polymerization and Functionalization Reactions

Thirty 7 cm×11 cm polyethylene pouches were prepared from 150 μm thick polyethylene tubing. The pouches were loaded with 5.74 mL of vinyl dimethylazlactone (VDM), 1.0 mL of a 0.25M solution of AIBN, and varying amounts of chain transfer agent (triethylsilane). Pouches 1-15 were further charged with one of several alcohols, 0.12 mL of DBU as a catalyst, and enough ethyl acetate to make the total volume up to 18 mL and then heat-sealed. Pouches 16-30 were loaded with 10.8-11.3 mL of ethyl acetate and a sealed 4 cm×6 cm polyethylene pouch (wall thickness 37.5 μm) containing the same amounts of alcohol and DBU as used above. They were then heat sealed. Thus the full library comprised two sub-libraries, a first one comprising samples 1-15 in which all the components of each sample shared one pouch, and a second sub-library, comprising samples 16-30, in which the components were split between a primary pouch and a captive pouch. The pouches were heated at 60° C. in a water bath overnight. The captive pouches were then ruptured by mechanical pressure on the outer pouch and all samples were returned to the 60° C. water bath for a further 12 hours.

T_{g}'s of the resultant alcohol-functionalized polymers were determined by DSC over the range of −50° C. to 200° C. and are shown in Table 11, below.

TABLE 11
All in one pouch

| | Triethyl- | | | Alcohol | DBU | |
|---|---|---|---|---|---|---|
| Sam- | silane | Ethyl acetate | | volume | volume | |
| ple | volume (mL) | volume (mL) | alcohol | (mL) | (mL) | Tg |
| 1 | 0 | 9.5 | MeOH | 1.65 | 0.12 | 134.2 |
| 2 | 0 | 7.4 | n-BuOH | 3.74 | 0.12 | 56.6 |
| 3 | 0 | 7.4 | s-BuOH | 3.75 | 0.12 | 176.7 |
| 4 | 0 | 5.7 | PGPE | 5.45 | 0.12 | 61.6 |
| 5 | 0 | 11.3 | none | | none | ** |
| 6 | 0.22 | 9.3 | MeOH | 1.65 | 0.12 | 125.4 |
| 7 | 0.22 | 7.2 | n-BuOH | 3.74 | 0.12 | 66.4 |
| 8 | 0.22 | 7.2 | s-BuOH | 3.75 | 0.12 | 168.2 |
| 9 | 0.22 | 5.5 | PGPE | 5.45 | 0.12 | 53.7 |
| 10 | 0.22 | 11.0 | none | | none | ** |
| 11 | 0.45 | 9.0 | MeOH | 1.65 | 0.12 | 126.8 |
| 12 | 0.45 | 7.0 | n-BuOH | 3.74 | 0.12 | 68.1 |
| 13 | 0.45 | 6.9 | s-BuOH | 3.75 | 0.12 | 162.1 |
| 14 | 0.45 | 5.2 | PGPE | 5.45 | 0.12 | 49.4 |
| 15 | 0.45 | 10.8 | none | | none | ** |

In two pouches

| Contents of primary pouch | | | Contents of captive pouch | | | Tg |
|---|---|---|---|---|---|---|
| 16 | 0 | 11.3 | MeOH | 1.65 | 0.12 | 117.4 |
| 17 | 0 | 11.3 | n-BuOH | 3.74 | 0.12 | 117.3 |
| 18 | 0 | 11.3 | s-BuOH | 3.75 | 0.12 | * |
| 19 | 0 | 11.3 | PGPE | 5.45 | 0.12 | 158.6 |
| 20 | 0 | 11.3 | None | | None | 172.7 |
| 21 | 0.22 | 11.0 | MeOH | 1.65 | 0.12 | 124.7 |
| 22 | 0.22 | 11.0 | n-BuOH | 3.74 | 0.12 | 128.0 |
| 23 | 0.22 | 11.0 | s-BuOH | 3.75 | 0.12 | 155.3 |
| 24 | 0.22 | 11.0 | PGPE | 5.45 | 0.12 | 155.1 |
| 25 | 0.22 | 11.0 | None | | none | 160.1 |
| 26 | 0.45 | 10.8 | MeOH | 1.65 | 0.12 | 121.7 |
| 27 | 0.45 | 10.8 | n-BuOH | 3.74 | 0.12 | 144.8 |
| 28 | 0.45 | 10.8 | s-BuOH | 3.75 | 0.12 | 158.6 |
| 29 | 0.45 | 10.8 | PGPE | 5.45 | 0.12 | 152.8 |
| 30 | 0.45 | 10.8 | None | | None | 159.1 |

*Not recorded because the inner pouch was not ruptured
**No T_{g} apparent

This example demonstrated the use of a captive pouch to ensure sequential, rather than parallel reaction with a nucleophile during the polymerization of a reactive monomer. The use of two pouches in the second sub-library ensured that reaction of the azlactone ring with the alcohol occurred after polymerization was essentially complete, while in the first sub-library the polymerization and ring-opening reactions occurred during overlapping time periods.

Various modifications and alterations of this invention will become apparent to those skilled in the art without departing from the scope and intent of this invention, and it should be understood that this invention is not to be unduly limited to the illustrative embodiments set forth herein.

1. A combinatorial array comprising fluid-impervious, flexible, self-supported pouches, each pouch comprising therein a member of a library of materials.

2. The array according to claim 1 wherein said pouches are selected from the group comprising polymeric films, metal foils, and composites of one or more of polymeric films, metal foils, paper products, woven textile materials, and non-woven textile materials.

3. The array according to claim 2 wherein said pouches are comprised of polymeric films.

4. The array according to claim 3 wherein said pouches are viscoelastic.

5. The array according to claim 1 wherein said pouches are sealable.

6. The array according to claim 1 wherein one or more of said pouches are sealed.

7. The array according to claim 1 wherein one or more of said pouches are transparent to actinic radiation.

8. The array according to claim 1 wherein said pouches are temporally spaced.

9. The array according to claim 1 wherein said materials comprise one or more of compounds, polymers, and blends and alloys of any of the foregoing.

10. The array according to claim 1 wherein said materials comprise biological species.

11. The array according to claim 1 wherein said materials are selected from the group consisting of solutions and emulsions.

12. The array according to claim 1 which is one or both of linearly and horizontally organized.

13. The array according to claim 1 comprising one or both of combinatorial chemical members and combinatorial physical members.

14. The array according to claim 1 wherein said pouches comprise a single layer film or multi-layer laminated film selected from the group consisting of homopolymers and copolymers of polyolefins, polydienes, polystyrenes, polyesters, polyethers, halogenated polyolefins, polyvinylalcohol, polyamides, polyimines, polycycloolefins, polyphosphazines, polyacetates and polyacrylates.

15. The array according to claim 1 wherein the pouches comprise metal foils comprising Group 2, 3, 4, 5, 6, 7 and 8 metals.

16. The array according to claim 1 wherein said pouches comprise composites one or more of polymer films, metal foils, paper materials, woven textile materials, non-woven textile materials, and biomaterials.

17. The array according to claim 1 wherein the members of said library are produced in macro-scale quantities.

18. A method for the synthesis of a combinatorial library of materials comprising the steps of:
a) providing a plurality of fluid-impervious, flexible, self-supporting pouches, each pouch containing therein one or more components for producing a member of a combinatorial library of materials, and
b) exposing said pouches to a controlled environment to cause said components to interact so as to produce said combinatorial library of materials.

19. The method according to claim 18 wherein said components are added one or both of simultaneously and sequentially.

20. The method according to claim 18 wherein one or more of said components are added after said pouch is sealed.

21. The method according to claim 20 wherein said components are added from one or more captive pouches.

22. The method according to claim 18 wherein said controlled environment further comprises added energy.

23. The method according to claim 22 wherein said added energy is selected from the group consisting of thermal, radiant, mechanical, and ultrasonic energy.

24. The method according to claim 18 wherein said interaction of components comprises a chemical reaction.

25. The method according to claim 24 wherein said chemical reaction is exothermic.

26. The method according to claim 18 wherein said interaction of components comprises a physical reaction.

27. The method according to claim 26 wherein said physical reaction comprises a blend or alloy of said components.

28. The method according to claim 18 wherein said library of materials is one or both of linearly and/or horizontally organized.

29. The method according to claim 28 wherein said library of materials are further labeled and archived.

30. The method according to claim 18 wherein said components are introduced into said pouches in an automated process.

31. The method according to claim 18 further comprising the step of analyzing said produced combinatorial library of materials in a non-destructive process.

32. The method according to claim 18 further comprising the step of analyzing said produced library of materials in a destructive process.

33. The method according to claim 14, wherein said materials are produced in macro-scale quantities.

34. The combinatorial library of products produced according to the method of claim 18.
